(19) United States
(12) Patent Application Publication
(10) Pub. No.: US 2012/0029577 A1
(43) Pub. Date: Feb. 2, 2012

(54) SYSTEM AND METHOD FOR BONE FIXATION USING BIODEGRADABLE SCREW HAVING RADIAL CUTOUTS

(76) Inventors: Sean Kerr, West Chester, PA (US); Brian Shultzabarger, West Chester, PA (US)

(21) Appl. No.: 13/191,789

(22) Filed: Jul. 27, 2011

Related U.S. Application Data
(60) Provisional application No. 61/368,277, filed on Jul. 28, 2010.

(51) Int. Cl. A61B 17/86; A61B 17/80; A61B 17/56

(52) U.S. Cl. 606/305; 606/311; 606/104; 606/286

(57) ABSTRACT

A system for bone fixation is provided including a biodegradable polymer screw and corresponding driver element. The screw is provided with a head having at least two regularly spaced notches. The driver element is provided with a distal end having at least two regularly spaced notches. The outer surface of the driver can correspond to the outer perimeter of the screw head and the notches and prongs are adapted to securably couple in a displacement fit to allow the drive to apply the screw into bone.

### CROSS REFERENCE TO RELATED APPLICATIONS

This application claims priority to U.S. Provisional Application Ser. No. 61/368,277, filed Jul. 28, 2010, the disclosure of which is hereby incorporated by reference in its entirety.

### FIELD OF THE DISCLOSURE

The present disclosure generally relates to biodegradable polymer screws and systems and methods for utilizing the screws for bone fixation procedures. In particular, the present disclosure relates to a biodegradable screw having radial cutouts in the screw head adapted to couple with a driver element having corresponding prongs that securably attach the screw in a displacement fit for insertion into bone.

### BACKGROUND

Biodegradable screws are becoming more prevalent in medical procedures because they can eliminate the need for a second removal operation after a first implantation operation, reduce stress shielding at the fixation site, reduce the opportunity for hardware migration and also reduce or eliminate post-operative artifact imaging.

Considerable forces are exerted on a screw during rotation of the screw as it is applied in bone fixation procedures. Where the screw head is shaped to include a centrally located recess to receive the drive element, these forces can deform the central recess of the biodegradable screw resulting in a loss of purchase between the screw head and the drive element (i.e., stripping). Additionally, even where the drive element remains engaged in the central recess, the rotational forces exerted on the polymer material that comprises the biodegradable screw may be so great as to shear the screw apart during insertion of the screw, leaving a fragmented screw shaft embedded in a bone fixation site. This can be especially true for extremely small or thin screws of the type commonly used in craniomaxillofacial procedures.

For example, when mandibular osteotomies are performed using conventional biodegradable screws, MMF (Maxillomandibular Fixation) devices are also typically used due to the lack of strength of the biodegradable screw. The MMF devices typically cause the patient's mandible be wired to the patient's maxilla for a period of time immediately following surgery. This MMF is not currently required when performing the procedure with traditional metallic screw fixation. Accordingly, the surgeon is disincentivized from using the conventional biodegradable screw over metallic fixation because of the additional procedure of wiring the jaw closed when using conventional polymeric screws.

What is therefore desired is an improved biodegradable screw.

### SUMMARY

Accordingly, the present disclosure relates to a system and method for bone fixation utilizing a biodegradable screw and a driver adapted to couple with the screw and insert the screw into an underlying bone. Any bone fixation procedure can be accomplished with the screw and driver disclosed herein, but particularly, bone fixation for craniofacial osteotomies, and more particularly for osteotomies related to orthognathic procedures involving the maxilla and mandible such as sagittal split osteotomies, vertical ramus osteotomies, inferior border osteotomies, sub apical osteotomies and genioplasties.

A biodegradable screw according to the present disclosure has a central axis and includes a head, shaft and distal end. The screw head has regularly spaced radial notches on its periphery for receiving a driver and distributing the forces of rotation away from a concentrated central point of the screw.

The present disclosure also relates to a driver for inserting the biodegradable screw into bone. The driver includes a driver body that defines a proximal end and a distal end, the driver body extending along a central axis from the proximal end to the distal end, and the driver body defining an outer surface. The proximal end is adapted to mate with a drive element, such as a handle, and a distal end that is adapted to couple with the biodegradable screw. The distal end of the driver has regularly spaced prongs spaced along the periphery of its distal end that can correspond to the notches of the screw head. In order to better relieve the stress placed on the material of the screw during rotation it is advantageous to place the notches on the periphery of the screw head rather than having a centrally located recess. By employing multiple prongs on the driver, the force exerted on the polymer material during application of the screw is more evenly distributed across the screw head. Even force distribution can be particularly desirable in small, thin screws typical in cranio-maxiofacial applications. The notches can couple with corresponding prongs from the driver in a unique secure displacement fit that prevents excess stress on the polymeric material of the screw head in the direction of rotation. In other words, the secure fit is accomplished by a displacement of the polymeric material of the screw head by the prongs in a direction normal to the direction of rotation. This displacement fit allows the screw to remain coupled to the driver permitting the surgeon to more easily apply the screw. A further advantage to the coupling is included where the outer surface of the distal end of the driver defines a first maximum cross-sectional dimension of the distal end of the driver and an outer perimeter of the screw head defines a second maximum cross-sectional dimension of the screw so that, according to one embodiment, the second maximum cross-sectional dimension is not less than the first maximum cross-sectional dimension when the notches and prongs are coupled in the secure displacement fit. This design allows the driver to fully apply the screw to a bone fixation site while preventing the outer surface of the driver from engaging bone and damaging the fixation site or over-widening the bone fixation site and possibly compromising the proper seating of the screw into the bone. It additionally prevents the driver from possible disruption of the bone fixation site or dislodging of the screw during withdrawal of the driver after the screw has been seated.

Additionally, the biodegradable screw can be provided with a central raised plateau on the screw head, located in an inner region of a proximal surface of the screw head from the notches. A corresponding recess located on the distal end of the driver can be sized to receive the raised plateau during coupling of the screw and the driver. When the distal end of the driver is placed proximally to and in contact with the screw head, the raised plateau acts as a self-centering mechanism by remaining within the prongs during axial rotation of the driver in instances where the prongs do not initially align with the corresponding notches in the screw head. By permitting the driver to remain centered on the screw head while a user rotates the driver to align the prongs with the notches, the raised plateau relieves the user from “forcing” the driver to remain in contact with the screw head with unnecessary application of axial force that could disrupt the polymeric material comprising the screw. Once the prongs and notches are in alignment, an axially directed force moves the driver distally with respect to the screw and engages the prongs and notches in the above-mentioned secure displacement while the central raised plateau is received within the recess.

Moreover, the screws disclosed herein can be treated to optimize the strength and rigidity of the polymer through a process called polymer orientation. There is occasionally a desire to utilize properties of polymers in applications where their strength and stiffness are not sufficient from conventional manufacturing methods such as injection molding, or machining of conventionally formed polymer stock. For example, a particular polymer may be desired as a bone screw due to its degradation profile and preferred bioaffinity but lacks the structural integrity necessary to withstand the forces encountered in such an application. In these cases, it may be advantageous to modify the polymer morphology from a spherulitic state, as is the case for a polymer that has cooled from the molten state, to a fibrillar (orientated) state. Increasing the yield strength and the elastic modulus becomes important as the use of polymer materials move from the role of a simple positioning device into areas of use where larger and larger forces are seen. In these load sharing and/or load bearing applications the conventionally formed polymers are simply not strong enough and therefore any product made from these methods can not be used. By realizing these increases, polymers and their advantages can be considered in load sharing and load bearing applications.

According to one embodiment of the disclosure the shaft of the biodegradable screw has an outer surface including a continuous helical threading. The shaft has a minor diameter and a major diameter. The threading has a proximal surface and a distal surface, and optionally a ridge. Alternatively, the shaft can have a non-continuous threading, or a series of protrusions oriented on the outer surface of the shaft in a generally helical pattern. In a preferred embodiment, the screw thread is adapted to be neither self-drilling, nor self-tapping, as those terms are understood in the art. In a more preferred embodiment, the threaded shaft is configured as a coarse buttress thread configuration.

According to another embodiment of the disclosure, the proximal end of the driver is adapted to couple in a standard hex coupling and in another embodiment the proximal end is adapted to couple in a snap-fit coupling to a ninety degree driving tool.

Further, a method of coupling the bone screw and the bone screw driver of the present disclosure includes:

a) centering the distal end of the driver over the proximal surface of a screw head such that the distal end of the driver is in physical contact with the screw head; and

b) applying an axially directed force to the driver such that the prongs engage with and couple to the notches in a secure displacement fit.

Where the screw includes the central raised plateau, the method can include the steps of:

a) centering the distal end of the driver over the proximal surface of a screw head such that the distal end of the driver is in physical contact with the screw head and the central raised plateau is maintained within the prongs of the driver;

b) axially rotating the driver while the central raised plateau is maintained within the prongs of the driver until the prongs are aligned with the notches; and

c) applying an axially directed force to the driver such that the prongs engage with and couple to the notches and the central raised plateau is received within the recess.

Additionally, a method for bone fixation is provided that includes the above disclosed steps of coupling the screw and driver and can optionally include the further steps of:

d) placing the distal tip of the screw at a bone fixation site;

e) axially rotating the driver to apply the screw into bone; and

f) disengaging the driver from the applied screw.

The method for bone fixation as described above can also optionally include placing a bone plate having a plurality of apertures at the fixation site and further include rotating the driver to apply the screw through one of the bone plate apertures into bone. In another embodiment of the above method, the screw is configured such that the method includes drilling at least one hole at the bone fixation site and threading (or tapping) the hole prior to applying the screw into the bone fixation site.

### BRIEF DESCRIPTION OF THE DRAWINGS

The foregoing summary, as well as the following detailed description of an example embodiment of the application, will be better understood when read in conjunction with the appended drawings, in which there is shown in the drawings an example embodiment for the purposes of illustration. It should be understood, however, that the application is not limited to the precise arrangements and instrumentalities shown. In the drawings:

FIG. 1 is a side elevation view of a biodegradable screw constructed in accordance with one embodiment;

FIG. 2 is a top plan view of the screw illustrated in FIG. 1;

FIG. 3 is perspective view of the screw illustrated in FIG. 1;

FIG. 4 is another perspective view of the screw illustrated in FIG. 1;

FIG. 5 is another top view of the screw illustrated in FIG. 1;

FIG. 6 is a sectional side elevation view of the screw taken along line 6-6 of FIG. 5;

FIG. 7 is a side elevation view of a driver constructed in accordance with one embodiment;

FIG. 8 is a perspective view of a distal end of the driver illustrated in FIG. 7;

FIG. 9 is a perspective view of the driver illustrated in FIG. 7;

FIG. 10 is a bottom plan view of the driver illustrated in FIG. 7;

FIG. 11 is a sectional side elevation view of the distal end of the driver taken along line 11-11 of FIG. 10;

FIG. 12 is a broken enlarged bottom plan view of a portion of the distal end of the driver at the dashed circle region illustrated in FIG. 10;

FIG. 13 is a perspective view of the distal end of the driver illustrated in FIG. 7;

FIG. 14 is another perspective view of the distal end of the driver illustrated in FIG. 7;

FIG. 15 is another bottom plan view of the driver illustrated in FIG. 7;

FIG. 16 is a side elevation view of a bone fixation system including the screw illustrated in FIG. 1 and the driver of FIG. 7, wherein the screw is illustrated in pre-engagement alignment with the driver;

FIG. 17 is a side elevation view of the bone fixation system illustrated in FIG. 16, wherein the is in a displacement fit with the driver;

FIG. 18 is a bottom plan view of the bone fixation system illustrated in FIG. 17;

FIG. 19 is a perspective view of the bone fixation system illustrated in FIG. 1, showing the screw being driven into a bone fixation site; and

FIG. 20 is a perspective view of the bone fixation system illustrated in FIG. 16, showing the screw being driven into a bone plate at the bone fixation site.

FIG. 21 is a perspective view of a driver constructed in accordance with an alternative embodiment;

FIG. 22 is a side elevation view of the driver illustrated in FIG. 16; and

FIG. 23 is a side view with partial cross-section of the distal end of the driver illustrated in FIG. 22.

### DETAILED DESCRIPTION

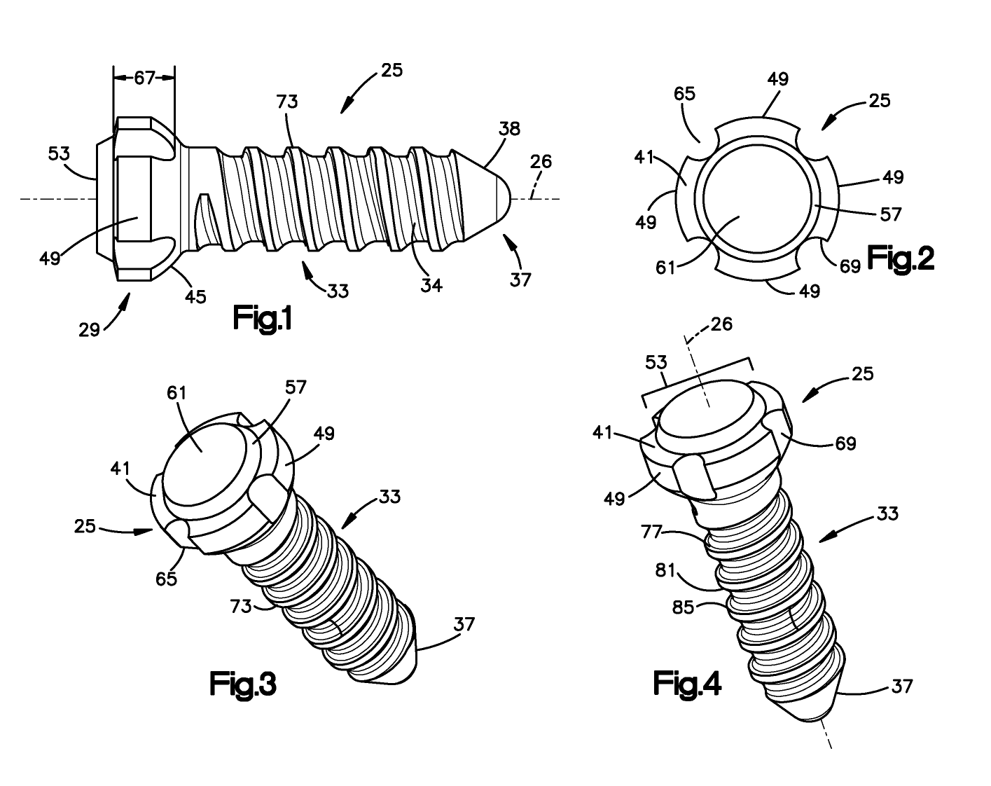

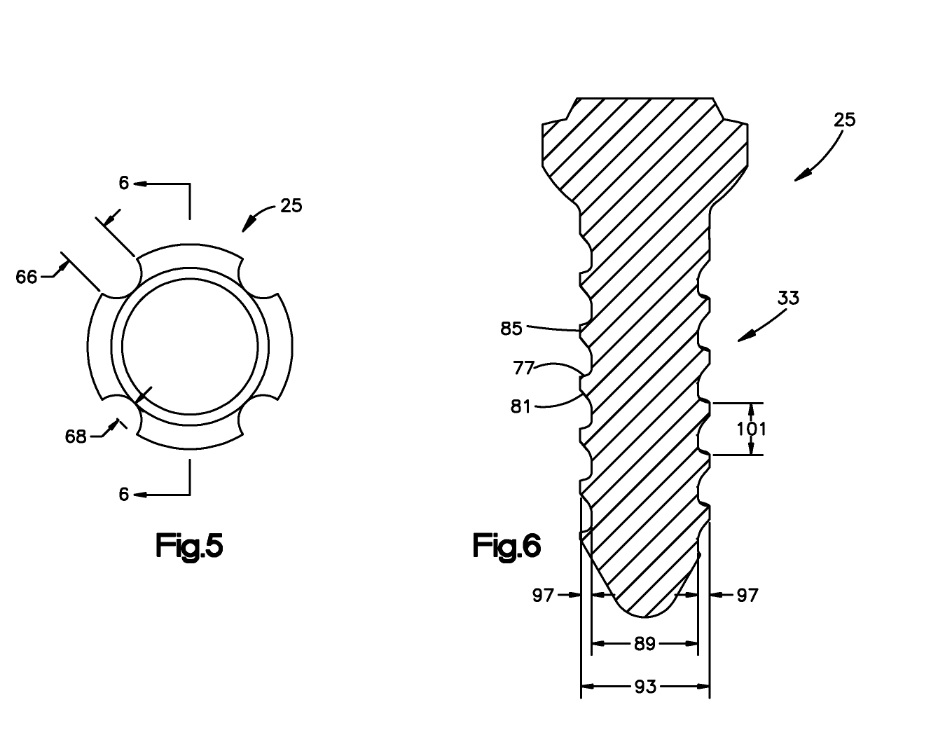

Referring to FIGS. 1-6, a biodegradable screw 25 includes a proximal head 29, a distal tip 37 axially opposed from the proximal head 29 along a central axis 26, and a shaft 33 that extends along the axis 26 between the head 29 and the distal tip 37. The screw 25 can be made from any suitable polymer, or polymeric blend; however, biodegradable polymers and/or blends thereof are the preferred starting material(s).

Biodegradable polymers contemplated as suitable for use as the starting material can include both homopolymers, and copolymers as wells as blends and combinations of both, such as polycaprolactone, polylactide, polyglycolide, poly(L-lactide), poly(D-lactide), poly(D,L-lactide), poly(L-lactide-co-D,L-lactide), poly(L-lactide-co-glycolide), poly(L-lactide-co-ε-caprolactone), poly(D,L-lactide-co-glycolide), poly(D,L-lactide-co-ε-caprolactone), polydioxanone and polycarbonates. In the case where the biodegradable polymer is a copolymer the monomer base unit ratio can be present in any range from 50:50 up to 96:4. Example biodegradable polymers are poly(L-lactide-co-glycolide) and poly(L-lactide-co-D,L-lactide). A preferred base unit range for poly(L-lactide-co-D,L-lactide) is 70:30 to 96:4. A preferred base unit range for poly(L-lactide-co-glycolide) is 80:20 to 90:10 and particularly preferred is 85:15.

Additionally, the screw 25 can be treated to optimize the strength and rigidity of the polymer through a known process called polymer orientation. Common methods of performing this change are a drawing operation, hydrostatic extrusion, and ram extrusion. All of these operations are mechanical operations that begin with a cross sectional area of polymer which is larger than the cross sectional area of the outlet of the process, commonly referred to as a die. In any of these processes, the draw ratio (ratio of beginning cross section to ending cross section) may be varied to impart different degrees of orientation into the polymer, and also ease in the processing. Another variable that may be used at some or all of the points in any of these operations is the application of heat. The vessels which contain the polymer may be heated. The die, the polymer itself, the ram, or any other part of this machinery may be heated to varying levels to impart different degrees of orientation to the polymer. Yet another factor in these processes is the force that is applied to the ending cross section after it has been drawn down; this force resists the natural tendency of the polymer to rebound during cooling to its original cross sectional shape and size. One skilled in the art can select any one of the above mentioned processes depending upon the characteristics of the preferred biodegradable polymer material.

With continuing reference to FIGS. 1-6, the head 29 of the screw 25 defines a proximal surface 41, a distal surface 45, and a side surface 49 that extends between the proximal surface 41 and the distal surface 45. The side surface 49 defines an outer perimeter of the head 29 and extends axially between proximal surface 41 and distal surface 45. The outer perimeter of head 29 can, according to one embodiment, define a maximum cross-sectional dimension of the screw. The proximal surface 41 extends substantially perpendicular to central axis 26 and can slope as desired either toward or away from the side surface 49 along a radial direction outward from the central axis 26. The distal surface 45 extends distally from the side surface 49 to the shaft 33. In accordance with the illustrated embodiment where the head 29 is represented as having a substantially circumferential outer perimeter, the diameter of the head 29 is greater than the major diameter 93 of the shaft 33. Accordingly, the distal surface 45 tapers radially inward from the side surface 49 towards the shaft 33 in a distal direction along the central axis 26, resulting in a head 29 configuration known in the art as a counter-sink. Other configurations are contemplated and depend upon the radial difference between the diameter of the head 29 and shaft 33 as well as the desired depth that screw 25 is intended to be driven into underlying bone.

According to one embodiment, the head 29 can also define an inner region having a central raised plateau 53. The central raised plateau 53 has a side wall 57 and a proximal face 61. The side wall 57 defines an outer perimeter of central raised plateau 53 and extends proximally from the proximal surface 41 to the proximal face 61. Side wall 57 can extend proximally substantially normal to the proximal surface 41 and alternatively can extend proximally from the proximal surface in a direction having an inward sloping radial component. The proximal face 61 extends radially in a direction substantially perpendicular to central axis 26.

The screw 25 also includes a plurality of (i.e., at least two) notches 65 that extend radially inward from the side surface 49 and are open at the side surface. The notches 65 are peripherally defined by an inner face 69 of the head 29 that extends into the side surface 49. The inner face 69 can be curved or rounded as illustrated, or can define any geometry as desired. The notches 65 have a height 67 extending distally from the proximal surface 53, through the side surfaces 49 towards the distal surface 45. The notches 65 also have a radial depth 68 extending radially inward from side surface 49, or otherwise stated toward the central axis 26. The notches 65 can terminate at the wall 57 of the central raised plateau 53 in accordance with the illustrated embodiment, however it should be appreciated that the notches can define any depth as desired. For instance, the notches 65 can terminate radially outward or inward of the wall 57. The notches 65 further have a cross-sectional width 66 that can decrease in a radial direction along the depth 68. Alternatively, the width 66 can increase or remain substantially constant in the radial direction along the depth 68. The width 66 can be constant along the height 67, or can increase or decrease along the proximal or distal direction.

The notches 65 can be spaced regularly along the periphery of head 29 as illustrated. For example, regular spacing of the notches can include spacing that is equidistant along the perimeter of head 29, as well as spacing that is equiangular such that at least two pairs of notches form equivalent central angles with respect to one another. Alternatively, one or more of the notches 65 can be spaced irregularly about the head 29. In accordance with the illustrated embodiment, the head 29 defines four notches 65 spaced ninety degrees apart from one another about the periphery of head 29. The head 29 has at least two notches 65 and preferably four, but can have any number based upon the physical properties of the biodegradable polymeric material used and the distribution of rotational forces that screw 25 will be subject to during application in bone.

With continuing reference to FIGS. 1-6, the shaft 33 of the screw 25 has an outer surface 34 that defines a minor diameter 89 measured radially through central axis 26. The shaft 33 extends distally along the central axis 26 from the distal surface 45 of head 29 to the distal tip 37. The shaft 33 is illustrated having a substantially cylindrical geometry (i.e., a constant minor diameter 89). It should be appreciated however that the shaft 33 can alternatively have a tapered configuration with a larger minor diameter 89 near the distal surface 45 of head 29 and a gradually decreasing minor diameter 89 as it extends towards the distal tip 37.

The outer surface 34 of the shaft 33 can include external threads 73. Threads 73 can be in a substantially continuous helical pattern or alternatively can be non-continuous or fragmented thread pattern. As another alternative, outer surface 34 may not include a thread, but rather a series of protrusions, for example teeth, that can either extend distally along the outer surface 34 in a generally helical pattern, or else in a linear or random distribution depending on the particular application or procedure that screw 25 is intended to be used. When the outer surface 34 of the shaft 33 contains threads 73 or some other type of protrusion, the shaft 33 will have a major diameter 93 measured as the radial distance of shaft 33 including threads 73. The threads 73 are illustrated in a continuous helical pattern, and include a proximal side 77 that faces the head 29, a distal side 81 that faces the distal tip 37, and can further include a ridge 85 that extends between the proximal side 77 and the distal side 81. The threads 73 further include a thread depth 97 that can be one-half the difference between the major diameter 93 and the minor diameter 89. The threads 73 further include a pitch 101 (or what is sometimes referred to as a lead) that is measured as the axial distance covered by threads 73 during one complete axial rotation of screw 25 and are typically categorized in the art as coarse threads for those with larger pitch lengths, and fine threads for those with smaller pitch lengths.

The threads 73 can be designed as desired, but most typical designs for use as metallic bone screws include self-drilling, and self-threading. In the embodiment illustrated in FIGS. 1-6, the threads 73 are configured as a non self-drilling, non-self-tapping configuration. The particular configuration illustrated is a coarse buttress thread design. A buttress thread configuration is known in the art and designed to withstand high axial load and high axial thrust in one direction making it well-suited for bone fixation and osteotomy procedures. In such a configuration, the proximal side 77 is the load bearing surface, oriented substantially perpendicular to the central axis 26, and extends from outer surface 34 to ridge 85 generally in an angular range of zero to twenty degrees with respect to the radial direction that extends perpendicular to the central axis 26. The ridge 85 extends substantially parallel to central axis 26, and the distal side 81 extends from ridge 85 back towards outer surface 34 generally in an angular range of thirty to sixty degrees with respect to the radial direction. Accordingly, the cross-sectional thread shape for a buttress design is illustrated as trapezoidal, which distinguishes a buttress design from those of self-drilling or self-tapping thread designs that are generally triangular in cross-section.

The distal tip 37 of the screw 25 has an outer surface 38. In accordance with the illustrated embodiment, the distal tip 37 is tapered being generally concave, wider where it meets the shaft 33 and gradually tapering inwards as it extends distally from the shaft 33. The distal tip 37 can also be designed as a blunt tip having a generally cylindrical or frusto-conical configuration, or a more pointed tip having a conical configuration.

It should be appreciated that the system of bone fixation described herein can include a plurality of screws 25 having various dimensional configurations according to the particular clinical indication and anatomical region to which they are intended to be used. For example, the screws 25 can have a range of lengths anywhere from about 6 mm up to about 100 mm, and for indications typical for craniomaxialfacial and orthognathic procedures, the screw lengths can be in the range of about 10 mm to about 18 mm. Additionally, the major diameter 93 of the screw 25 can have a range of diameters anywhere from about 1 mm to about 5 mm, and for indications typical for craniomaxialfacial and orthognathic procedures, the major diameter 93 of the screw 25 can have a range of about 2 mm to about 3 mm. It should be appreciated that these dimensions are provided as examples only, and the present disclosure is not intended to be limited to the dimensions provided.

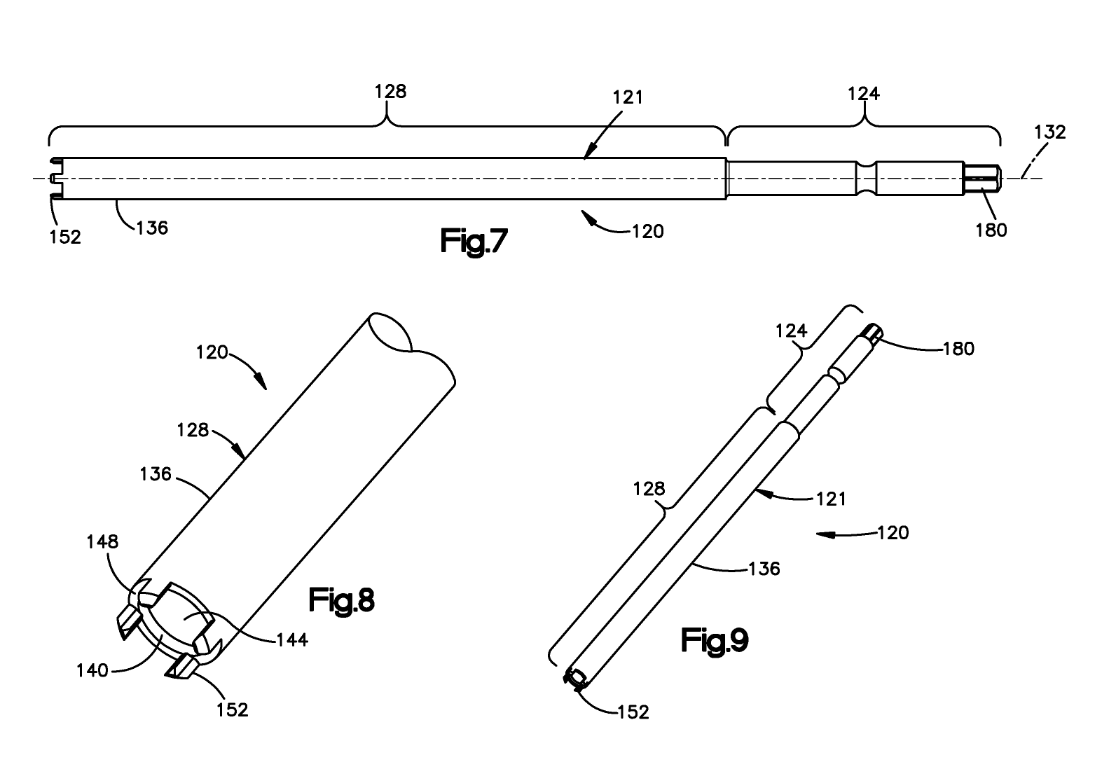

Referring now to FIGS. 7-15, a driver instrument 120, according to the system of bone fixation described herein, includes a driver body 121 that extends along a central axis 132, and defines a proximal end 124 and an axially opposed distal end 128. The proximal end 124 of the driver body 121 is adapted to engage a drive element or actuator, such as a handle, that imparts a rotational force to the driver instrument 120 so as to rotatably drive the driver instrument 120. The drive element can be manually or automatically actuated as desired. As illustrated in FIGS. 7 and 9, the proximal end 124 has a coupling 180 designed to be a male couple for a standard hex coupling engagement known in the art, though it should be appreciated that the coupling can be male or female and configured to mate with the drive element in any manner as desired.

The driver body 121 defines an outer surface 136 (which as illustrated is substantially circumferential) at the distal end 128, an inner surface 140 that is opposite to the outer surface 136 that defines a recess 144, and a distal surface 148 that can be axially directed between the inner and outer surfaces 136, 140. The driver 120 further includes prongs 152 that extend distally from the distal surface 148. The outer surface 136 extends axially along distal end 128 and defines an outer periphery of the driver body 121 at the distal end 128. The outer surface 136 further defines a first maximum cross-sectional dimension of the distal end of the driver and can define a maximum cross-sectional dimension of the prongs 152. The inner surface 140 extends axially along distal end 128 within and substantially parallel to outer surface 136. The inner surface 140 defines an outer periphery of the recess 144. The distal surface 148 extends radially between inner surface 140 and outer surface 136, and can extend perpendicular with respect to the axis 132, or can be sloped with respect to the axis 132 as desired.

A plurality of (i.e., at least two) prongs 152 extend distally from the distal end 128 and are spaced regularly apart from one another along distal surface 148, such that each prong can be aligned with a complementary notch 65 of the screw 25. According to one embodiment, there are an identical number of prongs and notches such that each prong 152 can couple with a complementary notch 65 of the head 29. In an alternative embodiment, there can be a greater number of notches 65 than prongs 152 such that there can be multiple complementary orientations of the prongs 152 with notches 65. In this type of embodiment, axial rotation of the driver 120 will permit multiple alignments where coupling of prongs 152 and notches 65 can occur.

Each prong 152 defines an inner face 168, and a radially opposed outer face 172. Each prong 152 further extends axially along a direction substantially parallel to the central axis 132 so as to define a height 156 extending axially from the distal surface 148, and depth 160 extending radially inward from the outer face 172 along a direction substantially perpendicular to the central axis 132. The outer face 172 of each prong 152 can be circumferential or alternatively shaped, and substantially continuous with the outer surface 136 of distal end 128. Otherwise stated, the outer face 172 can be aligned with the outer surface 136 such that the outer surface 136 and the outer face(s) 172 define an identical maximum cross-sectional dimension. Alternatively, the outer face 172 can be radially inwardly or outwardly offset with respect to the outer surface 136. Each of the prongs 152 defines a width 164 that can vary radially inward along the depth of the prong 152. For instance, in accordance with one embodiment, the width can be defined by a linear distance that extends between opposed radially outer ends of the inner face 168. In accordance with the illustrated embodiment, the width 164 decreases along its depth 160, for instance along the radially inward direction, though it should be appreciated that the width can remain constant or increase.

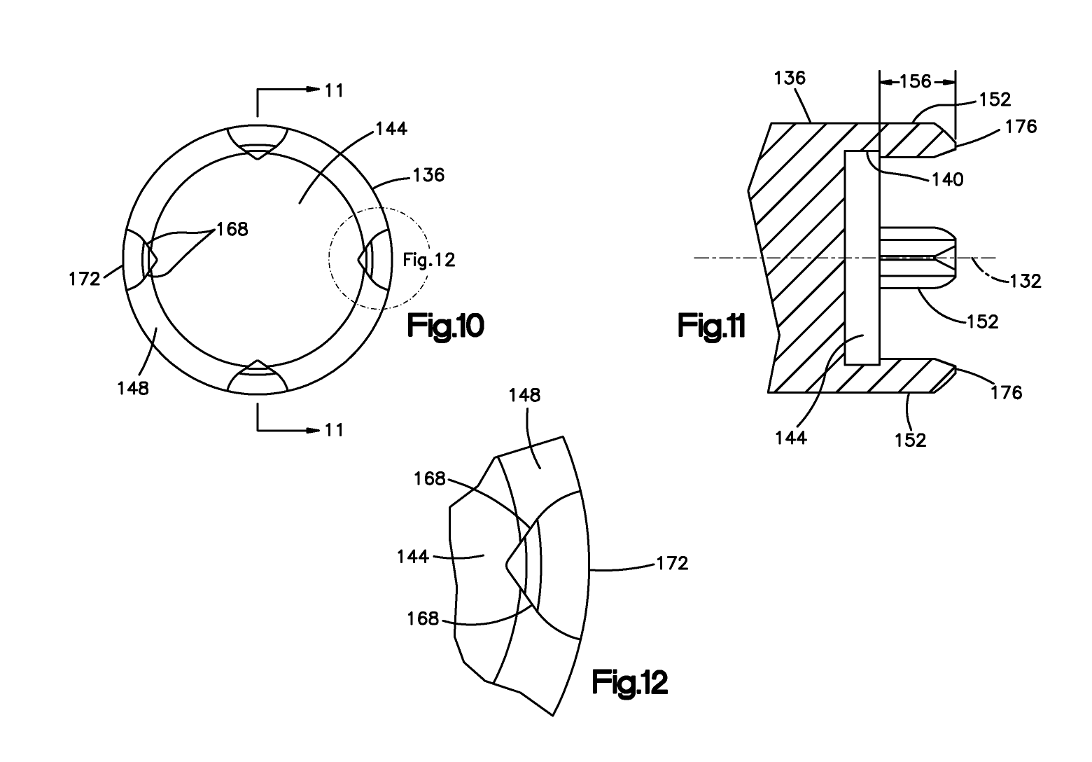

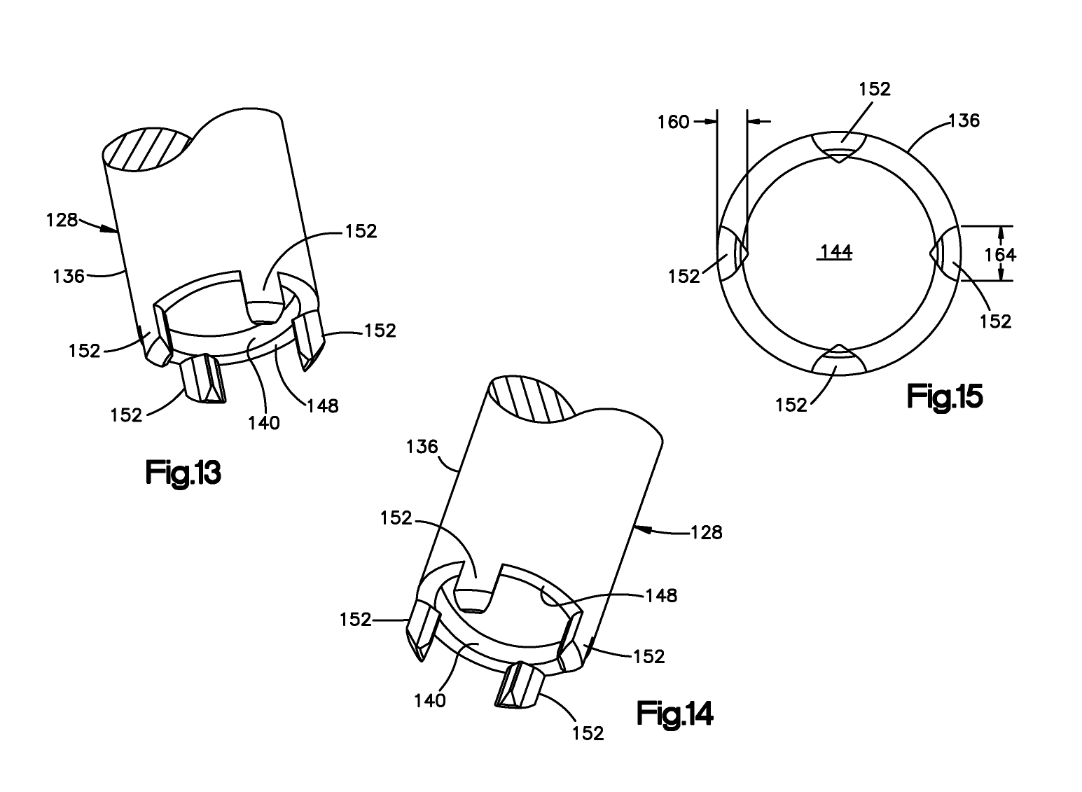

The inner face 168 can be shaped so as to correspond with the inner face 69 of the corresponding notch 65 of the screw 25 as described above. Thus, the inner face 168 can be shaped such that the prongs 152 have a radial cross-section that is substantially semicircular or can define the shape resembling a sector of a circle having defined by any angle as desired. Alternatively still, the inner face 168 can be shaped such that the radial cross-section can be substantially triangular or any geometry as desired so as to engage the screw head 29 in the complementary notches 65. As illustrated in FIGS. 10, 12 and 15, the inner face 168 has a shape such that prongs 152 have a blended semicircular/triangular radial cross-section wherein inner face 168 is shaped substantially semicircular near outer face 172 and as the depth 160 of prong 152 increases as it crosses through distal surface 148 the inner face 172 is shaped substantially planar such that the radial cross-section of prongs 152 assumes a more triangular configuration near recess 144. This particular configuration is best seen in FIG. 12. As shown in FIGS. 7-15, four prongs 152 are spaced regularly at ninety degree intervals along distal surface 148 and extend axially away from distal surface by height 156. While four prongs is a preferred embodiment, any number of two or more equiangular spaced prongs can be utilized depending upon the particular screw configuration driver 120 will be engaging.

The prongs 152 can further include a distal edge 176 formed at the distal most boundary of outer face 172 and inner face 168. In this embodiment, as best shown in FIGS. 11 and 22, outer face 172 slopes radially inward as it extends distally while inner face 168 slopes radially outward as it extends distally, thus forming edge 176. Thus, the distal edge 176 can further be referred to as a distal tip. The angle of slope for both the outer face 172 and inner face 168 can be variable and not necessarily the same for the faces. It should thus be appreciated that the slope of both the outer face 172 and the inner face 168 can be configured so as to accommodate the complementary geometry of the screw 25 to which driver 120 will couple. In accordance with one embodiment, the outer face 172 is sloped so as to properly align with a side surface 49 and tapered distal surface 45 of the screw head 29.

Referring now to FIGS. 16-20, a bone fixation system 123 includes the screw 25 and the driver 120 constructed as described herein. In particular, the distal end 128 of the driver 120 is configured (or adapted) to couple with the head 29 of the screw 25 such that the screw 25 is securely coupled to the driver 120 in a displacement fit that allows the driver 120 to implant the screw 25 into an underlying bone 190 at a bone fixation site 194.

In order to facilitate coupling between the driver 120 and the screw 25 according to one embodiment, the screw includes a plurality of notches 65 that are regularly spaced around the periphery of the head 29 while the driver 120 includes a plurality of regularly spaced prongs 152 at the distal end 128 along the distal surface 148 such that the regular spacing of notches 65 and prongs 152 permits an alignment of notches and prongs with each other. The outer surface 136 defines a first maximum cross-sectional dimension of distal end 128, including the prongs 152, while side surface 49 of the head 29 defines an outer perimeter of the head 29 which further defines a second maximum cross-sectional dimension of head 29 such that the second maximum cross-sectional dimension is not less than the first maximum cross-sectional dimension when the notches and prongs are coupled in the secure displacement fit.

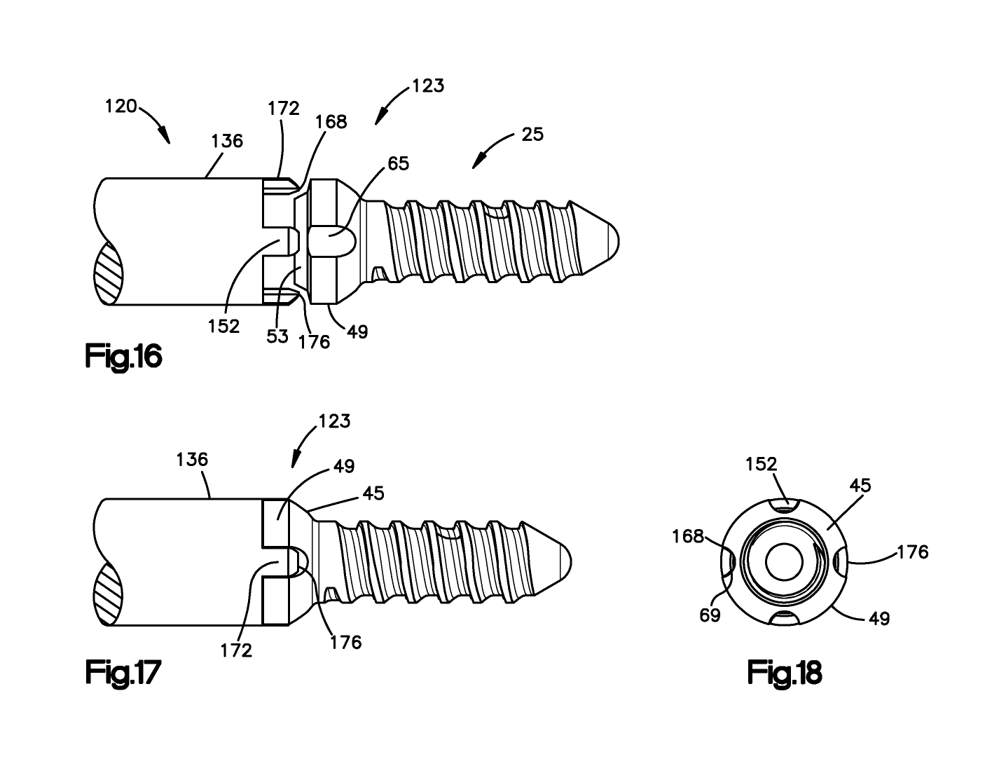

This can be seen in FIGS. 17-18 where the outer perimeter of head 29 defined by the side surface 49, the outer surface 136 of distal end 128, and the outer face 172 of prong 152 are in alignment, which as illustrated is a substantially circumferential alignment. It is also shown that the inner face 168 of the prong 152 is adjoined with the inner face 69 of the notch 65. A secure displacement fit between the screw 25 and the driver 120 occurs because the prong 152 has a first radial depth 160 that is greater than a second radial depth 68 of the notch 65. When coupled, the inner face 168 of the prong 152 applies a radially inwardly directed force (which is normal to the tangential force applied during rotation) to the inner face 60 of the notch 65, thereby causing displacement of the polymeric material in head 29. This displacement will secure head 29 of screw 25 to prongs 152 of driver 120. Additionally, as best shown in FIG. 17, the outer face 172 can be sloped along a portion of its length extending to the distal edge 176 to correspond to an equivalent slope of the distal surface 45 of the head 29.

Additionally, the distal end 128 of the driver 120 can also include a recess 144 that is defined by an inner surface 140. The recess 144 can be sized to accommodate the corresponding central raised plateau 53 of the screw 25, or stated another way, the plateau 53 can be sized to be received within the recess 144. This interface between the plateau 53 and the recess 144 provides a self-centering mechanism for the screw/driver coupling prior to the displacement fit of prongs 152 and notches 65. When the distal end 128 of the driver 120 is placed proximally to and in contact with the head 29 of the screw 25, there exists the possibility that the notches 65 will not be in alignment with the corresponding prongs 152. The central raised plateau 53 allows the prongs 152 remain in contact with the proximal surface 41 with the plateau 53 remaining within the prongs 152. This configuration allows a user to refrain from unnecessarily applying an axial force (and possibly damaging the polymeric material) in order to prevent the prongs 152 from slipping off of the head 29, which can allow the user to rotate driver 120 relative to the screw 25 to align the prongs 152 with the corresponding notches 65. When the prongs 152 are aligned with the notches 65, the user can then apply the necessary axial force to move the driver 120 distally and engage the prongs 152 into a secure displacement with the corresponding notches 65. The recess 144 is thus spaced to receive the central raised plateau 53 when the driver 120 moves distally relative to screw 25.

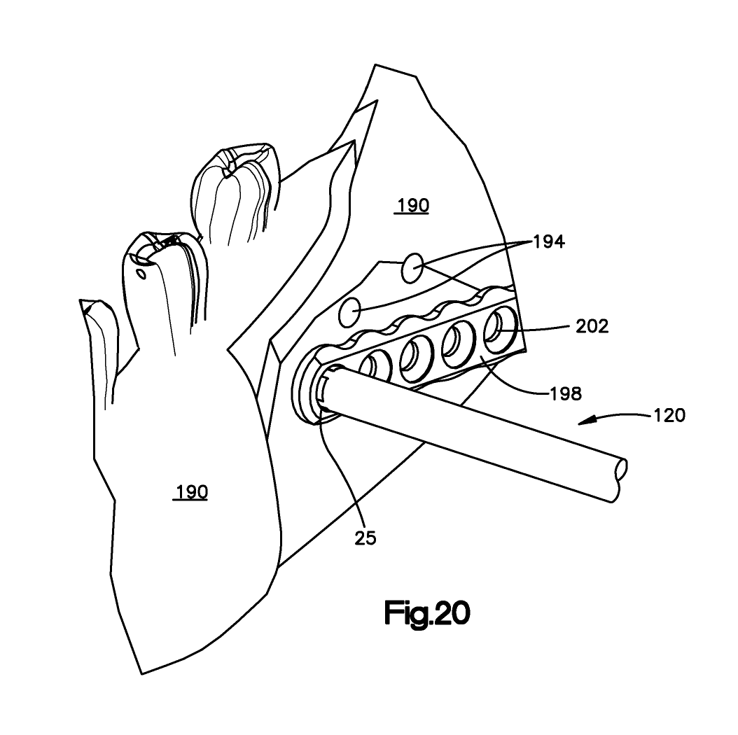

The bone fixation system 123 can also include at least one, including a plurality of bone plate(s) 198 having at least one apertures 202 therethrough an example of which is shown in FIG. 20. Such bone plates can be of any configuration suitable for the particular bone fixation procedure being performed. According to such an embodiment, the bone plate 198 is placed on a surface of the bone 190 at the bone fixation site 194 such that at least one of the apertures 202 of plate 198 is in alignment with fixation site 194 such that driver 120 can drive the screw 25 through the aperture 202 into fixation site 194 so as to fix the bone plate to underlying bone.

It should be appreciated that the bone fixation system 123 provides a method for coupling the bone screw 25 and driver 120 as well as the utilization of the system 123 for implanting the bone screw 25 into the underlying bone 190 at a target fixation site 194. While listed in a particular sequence, the following steps need not necessarily be performed in the exact manner as listed below. For example, a particular step in the method may be performed before, after, or simultaneously with another listed step of the method.

According to one embodiment, a method of coupling the screw and driver of the bone fixation system 123 can include the steps of:

a) centering the distal end of the driver over the proximal surface of a screw head such that the distal end of the driver is in physical contact with the screw head; and

b) applying an axially directed force to the driver such that the prongs engage with and couple to the notches in a secure displacement fit.

According to another embodiment, a method of coupling the screw and driver of the bone fixation system 123 can include the steps of:

a) centering the distal end of the driver over the proximal surface of a screw head such that the distal end of the driver is in physical contact with the screw head and the central raised plateau is maintained within the prongs of the driver;

b) axially rotating the driver while the central raised plateau is maintained within the prongs of the driver until the prongs are aligned with the notches; and

c) applying an axially directed force to the driver such that the prongs engage with and couple to the notches and the central raised plateau is received within the recess.

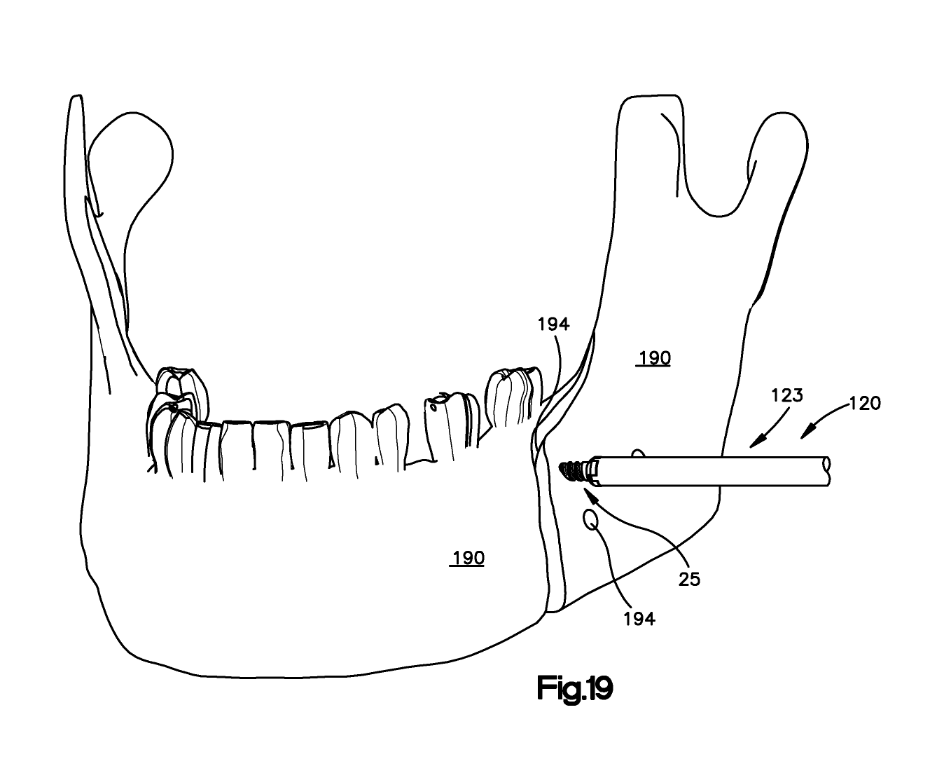

The bone fixation methods utilizing the system 123 disclosed herein can be performed on any malunion or non-union of bones or bone fragments both in vivo and ex vivo, on a human or on a non-human animal. One example method is for bone fixation following an osteotomy. As shown in FIGS. 19-20, a particular bone fixation method is for the repair of the mandible following a sagittal split osteotomy.

One example method of bone fixation includes carrying out the step previously identified to couple the bone screw and driver and further including the following steps:

d) placing the distal tip of the screw at a bone fixation site;

e) axially rotating the driver to apply the screw into bone; and

f) disengaging the driver from the applied screw;

Where the screw shaft and the distal tip are configured such that the screw 25 is not self-drilling, for example as a coarse buttress thread, the method can include the following step of drilling at least one bore hole into bone at a bone fixation site. Further, where the screw shaft and distal tip are configured such that the screw is not self-tapping (or self-threading), for example a coarse buttress thread, the method can include the step of tapping (or threading) the bore hole such that the bore hole can receive the particular thread pattern of the screw. Additionally, where the system includes a bone plate having at least one, or alternatively a plurality of apertures therethrough, the method can further include the steps of placing a bone plate at the surface of a bone fixation site, aligning the plate with bone such that at least one of the apertures is aligned with at least one bore hole in the bone, and axially rotating the driver to apply the screw through the aperture and into bone.

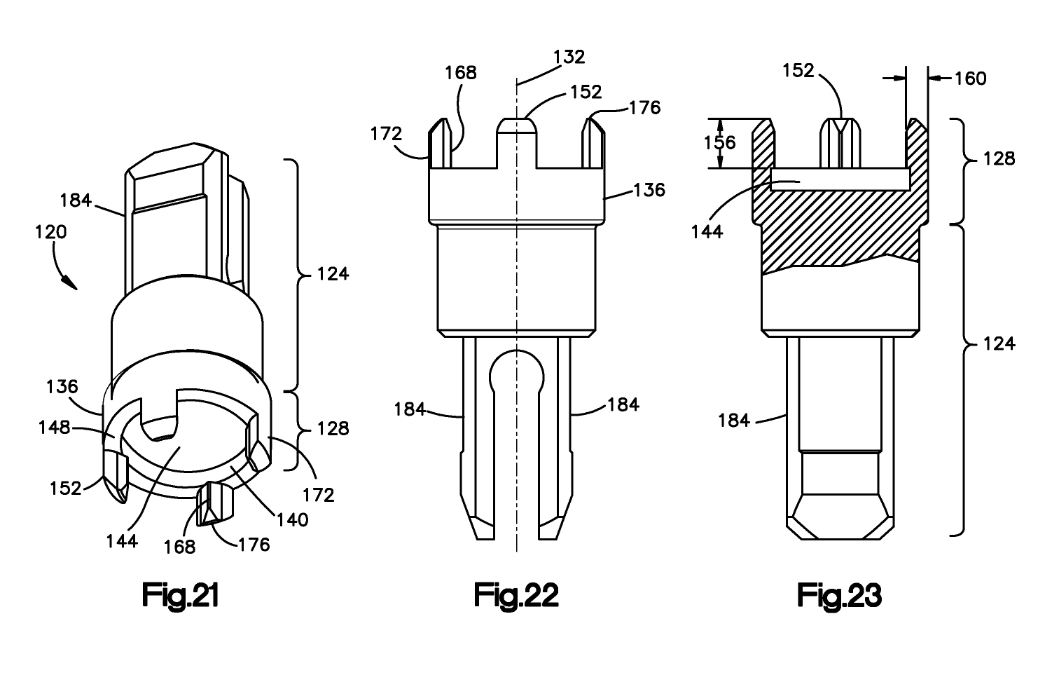

It should be appreciated that the bone fixation system 123 has been described in accordance with the illustrated screw 25 and driver 120, though it should be appreciated that the bone fixation system 123 and its components can be constructed in accordance with alternative embodiments without departing from the scope of the present disclosure, for instance as defined by the appended claims. For instance, referring now to FIGS. 21-23, the driver 120 is illustrated as described above, however the coupling 184 is configured as a male couple for a snap-fit engagement with a ninety-degree driver element known in the art.

Having described various embodiments of a system and method of bone fixation utilizing a biodegradable screw and corresponding driver, it is believed that other modifications, variations and changes will be appreciated by one skilled in the art in view of the teachings set forth in this disclosure. It is therefore understood that all such modifications, variations and changes would fall within the scope of the disclosure as defined in the appended claims.

What is claimed is:

1. A system for bone fixation comprising:
a bone screw comprised of a biodegradable polymer material, the bone screw having a head including a proximal surface, a distal surface and a side surface that extends between the proximal surface and the distal surface and further defines an outer perimeter of the screw head;
wherein the screw head defines at least two notches that extend distally along a direction from the proximal surface toward the distal surface, the notches are open at the side surface, and the notches are spaced apart from one another along the perimeter of the screw head.

2. The system of bone fixation of claim 1, wherein the notches of the bone screw are spaced regularly apart from one another along the perimeter of the screw head.

3. The system of bone fixation of claim 1, wherein the biodegradable polymer contains at least one polymer of the group consisting of: polycaprolactone, polylactide, polyglycolide, poly(L-lactide), poly(D-lactide), poly(D,L-lactide), poly(L-lactide-co-D,L-lactide), poly(L-lactide-co-glycolide), poly(L-lactide-co-ε-caprolactone), poly(D,L-lactide-co-glycolide), poly(D,L-lactide-co-ε-caprolactone), polydioxanone and polycarbonates.

4. The system of bone fixation of claim 3, wherein the biodegradable polymer is poly(L-lactide-co-glycolide) with a monomer base ratio in the range of about 70:30 to 90:10 of lactide to glycolide units.

5. The system of bone fixation of claim 3, wherein the biodegradable polymer is poly(L-lactide-co-D,L-lactide) with a monomer base ratio in the range of about 70:30 to 96:4 of L-lactide to D,L-lactide units.

6. The system of bone fixation of claim 3 wherein the biodegradable polymer has a polymer morphology in a fibrillar state.

7. The system of bone fixation of claim 1, wherein the screw head comprises at least four notches.

8. The system of bone fixation of claim 1, wherein the screw further comprises a threaded shaft that is neither self-drilling nor self-tapping.

9. The system of bone fixation of claim 1 further comprising:
a bone screw driver configured to impart a driving force onto the bone screw, the bone screw driver comprising:
a driver body that defines a proximal end and a distal end, the driver body extending along a central axis from the proximal end to the distal end;
the driver further comprising at least two prongs that extend distally from the distal end and are spaced apart from one another, the prongs adapted to couple with the notches of the bone screw so as to transfer a driving force to the bone screw.

10. The system of bone fixation of claim 9, wherein the driver body defines an outer surface, and an inner surface spaced radially inward from the outer surface along a direction substantially perpendicular to the central axis.

11. The system of bone fixation of claim 10, wherein the prongs have a first radial depth and the notches of the screw have a second radial depth, and the first radial depth is greater than the second radial depth before the notches couple to prongs, such that the prongs are adapted to couple with the notches of the screw head in a secure displacement fit.

12. The system of bone fixation of claim 11, wherein the outer surface of the driver defines a first maximum cross-sectional dimension at its distal end, the outer perimeter of the screw head defines a second maximum cross-sectional dimension, and the second maximum cross-sectional dimension is not less than the first maximum cross-sectional dimension when the notches and prongs are coupled in the secure displacement fit.

13. The system of bone fixation of claim 10, wherein the inner surface of the driver defines a recess, and the screw head further defines a centrally raised plateau sized to be received in the recess of the driver.

14. The system of bone fixation of claim 9, further comprising at least one bone plate that defines at least one aperture sized to receive the bone screw so as to fix the bone plate to underlying bone.

15. The system of bone fixation as recited in claim 1, further comprising a plurality of bone screws each comprised of a biodegradable polymer material, and each having a head including a proximal surface, a distal surface and a side surface that extends between the proximal surface and the distal surface and further defines an outer perimeter of the screw head;
wherein the screw head of each of the plurality of bone screws defines at least two notches that extend distally along a direction from the proximal surface toward the distal surface, the notches are open at the side surface, and the notches are spaced apart from one another along the perimeter of the screw head

16. The system of bone fixation of claim 15, further comprising at least one bone plate defining an aperture configured to receive at least one of the plurality of bone screws so as to fix the bone plate to underlying bone.

17. The system of bone fixation according to claim 9, wherein the distal end of the driver comprises at least four prongs, and wherein the screw head comprises at least four notches.

18. A method for coupling the bone screw and the bone screw driver of claim 10, the method comprising the steps of:
a) centering the distal end of the driver over the proximal surface of the screw head such that the distal end of the driver is in physical contact with the screw head;
b) applying an axially directed force to the driver such that the prongs engage with and couple to the notches in a secure displacement fit.

19. A method for coupling the bone screw and the bone screw driver of claim 13, the method comprising the steps of:
a) centering the distal end of the driver over the proximal surface of a screw head such that the distal end of the driver is in physical contact with the screw head and the central raised plateau is maintained within the prongs of the driver;
b) axially rotating the driver while the central raised plateau is maintained within the prongs of the driver until the prongs are aligned with the notches; and
c) applying an axially directed force to the driver such that the prongs engage with and couple to the notches and the central raised plateau is received within the recess.

20. The method for coupling the bone screw and the bone screw driver of claim 19, further comprising the steps of:
d) placing a distal tip of the screw at a bone fixation site;
e) axially rotating the driver to apply the screw into bone so as to fix the screw into the bone; and
f) disengaging the driver from the applied screw.
